(19) United States
(12) Patent Application Publication
(10) Pub. No.: US 2019/0240296 A1
(43) Pub. Date: Aug. 8, 2019

(54) TREATMENT OF DISEASES MEDIATED BY VASCULAR HYPERPERMEABILITY

(71) Applicant: AMPIO PHARMACEUTICALS, INC., Englewood, CO (US)

(72) Inventors: David BAR-OR, Englewood, CO (US); Gregory THOMAS, Highlands Ranch, CO (US)

(21) Appl. No.: 16/320,422

(22) PCT Filed: Jul. 26, 2017
(86) PCT No.: PCT/US2017/043856

§ 371 Date: Jan. 24, 2019

Related U.S. Application Data
(60) Provisional application No. 62/366,969, filed on Jul. 26, 2016.

(51) Int. Cl. A61K 38/38; A61P 27/02; A61K 45/06

(52) U.S. Cl. CPC A61K 38/385; A61K 2236/30; A61K 45/06; A61P 27/02

(57) ABSTRACT

The invention provides a method of inhibiting vascular hyperpermeability in an animal in need thereof. The method comprises administering an effective amount of a pharmaceutical composition prepared by removing albumin from a human serum albumin composition and one or more p38 MAPK inhibitors to the animal.

### CROSS-REFERENCE TO RELATED APPLICATIONS

This application claims the benefit of priority under 35 U.S.C. § 119(e) to U.S. Provisional Patent Application No. 62/366,969, filed Jul. 26, 2016. The entire disclosure of U.S. Provisional Patent Application No. 62/366,969 is incorporated herein by reference.

### FIELD OF THE INVENTION

The invention relates to a method and kit for inhibiting vascular hyperpermeability and the edema and other adverse effects that result from it. The method includes administering to an animal a low molecular weight fraction of human serum albumin (referred to as “LMWFHSA”) and a p38 MAPK inhibitor.

### BACKGROUND

The vascular endothelium lines the inside of all blood vessels. It acts as the interface between the blood and the tissues and organs. The endothelium forms a semi-permeable barrier that maintains the integrity of the blood fluid compartment, but permits passage of water, ions, small molecules, macromolecules and cells in a regulated manner. Dysregulation of this process produces vascular leakage into underlying tissues. Leakage of fluid into tissues causing edema can have serious and life threatening consequences in a variety of diseases. Accordingly, it would be highly desirable to have a method for reducing edema, preferably at its earliest stage, and restoring the endothelial barrier to physiological.

Breakdown of the inner blood-retinal barrier is a contributing factor in the pathogenesis of several ocular diseases including diabetic retinopathy, age-related macular degeneration, and retinal vein occlusion (Klaassen I, et al. Molecular basis of the inner blood-retinal barrier and its breakdown in diabetic macular edema and other pathological conditions. Progress in retinal and eye research 2013; 34:19-48). The resulting accumulation of fluid and protein thickens the macula, impairing visual acuity. If left unchecked, macular edema can lead to permanent vision loss and is the primary cause of blindness in diabetes. In 2012, an estimated 29.1 million Americans suffered from diabetes with an additional 86 million exhibiting signs of prediabetes (National Diabetes Statistics Report: Estimates of Diabetes and Its Burden in the United States. In: Services UDoHaH (ed). Atlanta, Ga.: Centers for Disease Control and Prevention; 2014). Taken together with an aging population, this condition poses a serious health risk for the industrial world.

The loss of endothelial barrier integrity under pathologic conditions is primarily the result contractile forces exerted by the actin cytoskeleton. Pro-inflammatory mediators trigger second messenger systems that activate both myosin light chain kinase and Rho-associated coiled coil-containing protein kinase which promote the phosphorylation-dependent binding of myosin II motors to f-actin stress fiber bundles (Dudek S M, Garcia J G. Cytoskeletal regulation of pulmonary vascular permeability. Journal of applied physiology 2001; 91:1487-1500). In tandem or independently, actin cytoskeletal remodeling can also be regulated by p38 MAPK. Under normal physiologic conditions, 27-kDa heat shock protein sequesters actin monomers in the cytosol but its phosphorylation by p38 MAPK relieves this constraint, freeing actin to participate in stress fiber formation (Mehta D, Malik A B. Signaling mechanisms regulating endothelial permeability. Physiological reviews 2006; 86:279-367). Because endothelial cell contractile machinery is anchored to both cell junctions and focal adhesions, the resulting actomyosin tension physically pulls open gaps in the monolayer.

A strong body of evidence suggests that the microtubule network also helps govern paracellular permeability. For example, incubation of endothelial cells with paclitaxel, a microtubule stabilizing compound, significantly reduces TNFα-induced permeability (Petrache I, et al. The role of the microtubules in tumor necrosis factor-alpha-induced endothelial cell permeability. American journal of respiratory cell and molecular biology 2003;28:574-581). Conversely, destabilization of microtubules with nocodazole and vinblastine increases permeability through myosin light chain phosphorylation and Rho-GTPase activation (Verin A D, Birukova A, Wang P, et al. Microtubule disassembly increases endothelial cell barrier dysfunction: role of MLC phosphorylation. American journal of physiology Lung cellular and molecular physiology 2001; 281:L565-574). Interestingly, the disruption of microtubules with 2-methoxyestradiol is attenuated by treatment with the p38 inhibitor, SB203580 (Bogatcheva N V, Adyshev D, Mambetsariev B, Moldobaeva N, Verin AD. Involvement of microtubules, p38, and Rho kinases pathway in 2-methoxyestradiol-induced lung vascular barrier dysfunction. American journal of physiology Lung cellular and molecular physiology 2007; 292:L487-499). As a whole, these findings indicate that an intimate relationship exists, across all components of the cytoskeleton, to regulate permeability.

Transport directly across the endothelium by the process known as transcytosis contribute to barrier function as well. This mode of action is of particular interest to immunologically privileged compartments such as the eye and for the passage of macromolecules. Shuttling begins as caveolae or “cave-like” invaginations form on the apical plasma membrane, trapping luminal solutes and receptor bound ligands in endocytic vesicles (Yuan S Y, Rigor R R. Regulation of Endothelial Barrier Function. San Rafael (CA); 2010). These vesicles can then recycle back to the apical membrane or pass through the interior of the cell to release their contents by exocytosis on the basolateral side. Disruption of microtubules reduces the surface level of caveolae on endothelial cells, demonstrating a dependence of the cytoskeleton to this pathway (Mehta D, Malik A B. Signaling mechanisms regulating endothelial permeability. Physiological reviews 2006; 86:279-367). The passage of solutes by this route cannot be underestimated for the fluid volume residing within caveolae constitutes 15-20% of the interior volume of endothelial cells (Yuan S Y, Rigor R R. Regulation of Endothelial Barrier Function. San Rafael (CA); 2010).

### SUMMARY OF THE INVENTION

One embodiment of the invention relates to a method of inhibiting vascular hyperpermeability in an animal in need thereof comprising administering to the animal an effective amount of a pharmaceutical composition comprising prepared by removing albumin from a human serum albumin composition; and one or more p38 MAPK inhibitors.

In one aspect, the one or more p38 MAPK inhibitors is selected from the group consisting of SB 203580, SB 203580 hydrochloride, SB 202190, SB 239063, SB 706504, AL 8697, AMG 548, CMPD-1, DBM 1285 dihydrochloride, EO 1428, JX 401, ML 3403, RWJ 67657, SCIO 469 hydrochloride, SKF 86002 dihydrochloride, SX 011, TA 01, TA 02, TAK 715, VX 702, VX 745, p38 MAPK Inhibitor TOCRISET™, and combinations thereof.

In one aspect, the animal has a disease or condition mediated by vascular hyperpermeability.

In one aspect, administration of the pharmaceutical composition and the one or more p38 MAPK inhibitors is commenced immediately upon diagnosis of the disease or condition.

In one aspect, the disease or condition is an ocular disease.

In yet another aspect, the disease or condition is a vascular complication of diabetes.

In still another aspect, the disease or condition is a vascular complication of diabetes. For example, the vascular complication is edema, accumulation of low density lipoproteins in subendothelial space, accelerated atherosclerosis, accelerated aging of vessel walls in the brain, myocardial edema, myocardial fibrosis, diastolic dysfunction, diabetic cardiomyopathy, retardation of lung development in the fetuses of diabetic mothers, alterations of one or more pulmonary physiological parameters, increased susceptibility to infections, vascular hyperplasy in the mesentery, diabetic neuropathy, diabetic macular edema, diabetic nephropathy, diabetic retinopathy, or redness, discoloration, dryness and ulcerations of the skin. In one aspect, the vascular complication is edema. In another aspect, the vascular complication is diabetic cardiomyopathy. In yet another aspect, the vascular complication is diabetic neuropathy. In still another aspect, the vascular complication is diabetic macular edema. In yet another aspect, the vascular complication is diabetic retinopathy. In one aspect, the diabetic retinopathy is nonproliferative diabetic retinopathy. In still another aspect, the vascular complication is diabetic nephropathy.

In still another aspect, the disease or condition is an acute lung injury, acute respiratory distress syndrome, age-related macular degeneration, atherosclerosis, choroidal edema, choroiditis, coronary microvascular disease, cerebral microvascular disease, diabetes, Eals disease, edema caused by injury, edema associated with hypertension, glomerular vascular leakage, hemorrhagic shock, hypertension, Irvine Gass Syndrome, ischemia, macular edema, nephritis, nephropathies, nephrotic edema, nephrotic syndrome, neuropathy, organ failure due to edema, pre-eclampsia, pulmonary edema, pulmonary hypertension, renal failure, retinal edema, retinal hemorrhage, retinal vein occlusion, retinitis, retinopathy, silent cerebral infarction, systemic inflammatory response syndrome, transplant glomerulopathy, uveitis, vascular leakage syndrome, vitreous hemorrhage or Von Hipple Lindau disease. In one aspect, the disease or condition is macular edema. In yet another aspect, the disease or condition is a neuropathy. In still another aspect, the disease or condition is a retinopathy.

In another aspect, the animal is in need of the of the pharmaceutical composition and one or more p38 MAPK inhibitors, because of one or more early signs of, or a predisposition to develop, a disease or condition mediated by vascular hyperpermeability. In one aspect, the disease or condition is diabetes, hypertension, atherosclerosis or an ocular disease.

In yet another aspect, the vascular hyperpermeability is vascular hyperpermeability of a continuous endothelium found in, or around, a brain, diaphragm, duodenal musculature, fat, heart, kidney, large blood vessel, lung, mesentery, nerve, retina, skeletal muscle, skin or testis. In one aspect, the continuous endothelium is found in, or around, a brain, heart, lung, nerve or retina.

In still another aspect, the vascular hyperpermeability is vascular hyperpermeability of a fenestrated endothelium found in, or around, a kidney, a pancreas, an adrenal, an endocrine gland or an intestine. In one aspect, the fenestrated endothelium is found in a kidney.

In still another aspect, the step of removing the albumin comprises treating the human serum albumin composition by a separation method selected from the group consisting of ultrafiltration, sucrose gradient centrifugation, chromatography, salt precipitation, and sonication. In yet another aspect, the step of removing comprises passing the human serum albumin composition over an ultrafiltration membrane with a molecular weight cut off that retains the albumin, and wherein the resulting filtrate comprises LMWFHSA.

Another embodiment of the invention is a pharmaceutical composition prepared by removing albumin from a human serum albumin composition and one or more p38 MAPK inhibitors for the treatment of a disease or condition mediated by vascular hyperpermeability and/or for the inhibition of vascular hyperpermeability. In one aspect the disease or condition is an ocular disease.

In one aspect, the one or more p38 MAPK inhibitors is selected from the group consisting of SB 203580, SB 203580 hydrochloride, SB 202190, SB 239063, SB 706504, AL 8697, AMG 548, CMPD-1, DBM 1285 dihydrochloride, EO 1428, JX 401, ML 3403, RWJ 67657, SCIO 469 hydrochloride, SKF 86002 dihydrochloride, SX 011, TA 01, TA 02, TAK 715, VX 702, VX 745, p38 MAPK Inhibitor TOCRISET™, and combinations thereof.

### BRIEF DESCRIPTION OF THE DRAWINGS

FIGS. 1A and 1B. A composition prepared by removing albumin from a human serum albumin composition, i.e., a LMWFHSA, reduces macromolecular permeability and late phase barrier disruption in human retinal endothelial cells (HREC). (FIG. 1A) HREC were grown to confluence on transwell inserts then treated with saline, LMWF5A, or 10 μM Forskolin. Macromolecular permeability over 24 hours was measured by HRP and relative permeability versus saline controls calculated. Data presented as Mean±SD (One-way ANOVA followed by Bonferroni's post hoc, *=p<0.025, n=3) (FIG. 1B) HREC were grown to confluence on electrode arrays then trans-endothelial resistance monitored for 48 hours following treatment with saline or LMWF5A.

FIGS. 2A-2C. LMWF5A induces the acetylation of α-tubulin in HREC. (FIG. 2A) Immunofluorescence staining for acetylated α-tubulin in HREC treated with saline or LMWF5A for 3 hours. (FIG. 2B) Quantification performed for representative immunofluorescence experiment. HREC were treated for 0.5, 3, 6, or 24 hours and stained for acetylated α-tubulin. Data presented as mean FU normalized to number of cells determined by DAPI counter staining +SD (One-way ANOVA followed by Bonferroni's post hoc, *=p<0.01 versus Saline, **=p<0.02 versus 3 hr LWWFSA, n=6). (FIG. 2C) Representative western blot performed for acetylated α-tubulin on lysates from HREC treated with LMWF5A for 0.5, 3, 6, or 24 hours. Densitometry was then performed and normalized to actin loading.

FIG. 3: LMWF5A induced changes in the localization of acetylated α-tubulin in HREC. Representative immunofluorescence staining for acetylated α-tubulin in HREC treated with saline or LMWF5A for 3, 6, or 24 hours. Acetylated α-tubulin in saline treated controls is primarily located in microtubule organizing centers around the nucleus. LMWF5A treated HREC exhibit elevated cytoplasmic and perinuclear staining.

FIGS. 4A and 4B. Inhibition of PI3-kinase reduces while inhibition of p38 MAPK potentiates LMWF5A induced acetylation of acetylated α-tubulin. (FIG. 4A) Representative immunofluorescence of HREC exposed to LMWF5A for 3 hours in the presence of specific inhibitors to PI3-kinase (10 μM LY294002) or p38 MAPK (10 μM SB203580). Data presented as mean FU normalized to number of cells determined by DAPI counter staining±SD (One-way ANOVA followed by Bonferroni's post hoc, *=p<0.01 versus Saline±DMSO, **=p<0.025 versus LWWF5A±DMSO, n=6). (FIG. 4B) Representative western blot performed for acetylated α-tubulin on lysates from HREC treated with LMWF5A for 3 hours in the presence of specific inhibitors. Densitometry was then performed and normalized to actin loading.

### DETAILED DESCRIPTION OF THE OF THE INVENTION

The present invention provides a method of inhibiting vascular hyperpermeability. The method comprises administering an effective amount of a pharmaceutical composition comprising a low molecular weight fraction of human serum albumin (LMWFHSA) and one or more p38 mitogen-activated protein kinase (MAPK) inhibitors to an individual having a need thereof.

The invention also provides for a pharmaceutical composition comprising LMWFHSA and one or more p38 MAPK inhibitors for the treatment of a disease or condition mediated by vascular hyperpermeability and/or for the inhibition of vascular hyperpermeability.

Inhibition of vascular hyperpermeability according to the invention includes inhibition of paracellular-caused hyperpermeability and transcytosis-caused hyperpermeability. Recent evidence indicates that transcytosis-caused hyperpermeability is the first step of a process that ultimately leads to tissue and organ damage in many diseases and conditions. Accordingly, the present invention provides a means of early intervention in these diseases and conditions which can reduce, delay or even potentially prevent the tissue and organ damage seen in them.

The invention also provides a method of modulating the cytoskeleton of endothelial cells in an animal. The method comprises administering an effective amount of an active ingredient, wherein the active ingredient comprises LMWFHSA, or a pharmaceutically-acceptable salt thereof, and one or more p38 MAPK inhibitors to the animal.

The invention further provides a kit. The kit comprises LMWFHSA, or a pharmaceutically-acceptable salt thereof and one or more p38 MAPK inhibitors.

Described in more detail below are the unexpected effects of LMWFHSA and one or more p38 MAPK inhibitors on endothelial permeability. A LMWFHSA-containing composition as disclosed herein (also referred to LMWF5A) can be a biologic derived from the less than 5 kDa fraction of human serum albumin. In clinical trials, a single intra-articular injection of LMWFHSA resulted in a significant 42.3% reduction in pain observed 4 weeks following injection that persisted to the completion of the trial versus saline controls (Bar-Or D, et al. A randomized clinical trial to evaluate two doses of an intra-articular injection of LMWFHSA in adults with pain due to osteoarthritis of the knee. PloS one 2014; 9:e87910). In vitro experiments have also demonstrated that this LMWFHSA possesses anti-inflammatory properties by inhibiting cytokine release from both stimulated peripheral blood mononuclear cells (PBMC) and T-cell lines (Bar-Or D, Thomas G W, Bar-Or R, et al. Commercial human albumin preparations for clinical use are immunosuppressive in vitro. Critical care medicine 2006; 34:1707-1712; Shimonkevitz R, et al. A diketopiperazine-containing fraction of human serum albumin modulates T-lymphocyte cytokine production through rapl. The Journal of trauma 2008; 64:35-41; Thomas G W, et al. Anti-Inflammatory Activity in the Low Molecular Weight Fraction of Commercial Human Serum Albumin (LMWF5A). J Immunoassay Immunochem 2016; 37:55-67). Recent studies show that LMWFHSA potentiates the release of the anti-inflammatory prostaglandin, 15d-PGJ2 from LPS stimulated PBMC as well (Thomas GW, et al. Anti-Inflammatory Activity in the Low Molecular Weight Fraction of Commercial Human Serum Albumin (LMWF5A). J Immunoassay Immunochem 2016; 37:55-67; Thomas G W, Rael L T, Hausburg M, et al. The low molecular weight fraction of human serum albumin upregulates production of 15d-PGJ in Peripheral Blood Mononuclear Cells. Biochem Biophys Res Commun 2016). Furthermore, LMWFHSA treatment of bone marrow derived mesenchymal stem cells reduces Rho GTPase activity and stress-fiber formation (Bar-Or D, et al. Low Molecular Weight Fraction of Commercial Human Serum Albumin Induces Morphologic and Transcriptional Changes of Bone Marrow-Derived Mesenchymal Stem Cells.Stem Cells Transl Med 2015; 4:945-955).

As discussed in more detail below, by studying both in vitro permeability as well as monitored cytoskeletal changes by both cell imaging and immunoblot it has been unexpectedly found that LMWFHSA reduces permeability and mediates changes to the microtubule network. These observations expand the knowledge of the biologic activities surrounding LMWFHSA and in particular, and suggest that this biologic alters microtubule dynamics and transcytosis.

“Vascular hyperpermeability” is used herein to mean permeability of a vascular endothelium that is increased as compared to basal levels. “Vascular hyperpermeability,” as used herein, includes paracellular-caused hyperpermeability and transcytosis-caused hyperpermeability.

“Paracellular-caused hyperpermeability” is used herein to mean vascular hyperpermeability caused by paracellular transport that is increased as compared to basal levels. Other features of “paracellular-caused hyperpermeability” are described below.

“Paracellular transport” is used herein to mean the movement of ions, molecules and fluids through the interendothelial junctions (IEJs) between the endothelial cells of an endothelium.

“Transcytosis-caused hyperpermeability” is used herein to mean vascular hyperpermeability caused by transcytosis that is increased as compared to basal levels.

“Transcytosis” is used herein to mean the active transport of macromolecules and accompanying fluid-phase plasma constituents across the endothelial cells of the endothelium. Other features of “transcytosis” are described below.

“Basal level” is used herein to refer to the level found in a normal tissue or organ.

“Inhibiting, “inhibit” and similar terms are used herein to mean to reduce, delay or prevent.

An animal is “in need of” treatment according to the invention if the animal presently has a disease or condition mediated by vascular hyperpermeability, exhibits early signs of such a disease or condition, or has a predisposition to develop such a disease or condition.

“Mediated” and similar terms are used here to mean caused by, causing, involving or exacerbated by, vascular hyperpermeability.

The endothelium is a key gatekeeper controlling the exchange of molecules from the blood to the tissue parenchyma. It largely controls the permeability of a particular vascular bed to blood-borne molecules. The permeability and selectivity of the endothelial cell barrier is strongly dependent on the structure and type of endothelium lining the microvasculature in different vascular beds. Endothelial cells lining the microvascular beds of different organs exhibit structural differentiation that can be grouped into three primary morphologic categories: sinusoidal, fenestrated and continuous.

Sinusoidal endothelium (also referred to as “discontinuous endothelium”) has large intercellular and intracellular gaps and no basement membrane, allowing for minimally restricted transport of molecules from the capillary lumen into the tissue and vice versa. Sinusoidal endothelium is found in liver, spleen and bone marrow.

Fenestrated endothelia are characterized by the presence of a large number of circular transcellular openings called fenestrae with a diameter of 60 to 80 nm. Fenestrated endothelia are found in tissues and organs that require rapid exchange of small molecules, including kidney (glomeruli, peritubular capillaries and ascending vasa recta), pancreas, adrenal glands, endocrine glands and intestine. The fenestrae are covered by thin diaphragms, except for those in mature, healthy glomeruli. See Ichimura et al., J. Am. Soc. Nephrol., 19: 1463-1471 (2008).

Continuous endothelia do not contain fenestrae or large gaps. Instead, continuous endothelia are characterized by an uninterrupted endothelial cell monolayer. Most endothelia in the body are continuous endothelia, and continuous endothelium is found in, or around, the brain (blood brain barrier), diaphragm, duodenal musculature, fat, heart, some areas of the kidneys (papillary microvasculature, descending vasa recta), large blood vessels, lungs, mesentery, nerves, retina (blood retinal barrier), skeletal muscle, testis and other tissues and organs of the body.

Endothelial transport in continuous endothelium can be thought of in a general sense as occurring by paracellular and transcellular pathways. The paracellular pathway is the pathway between endothelial cells, through the interendothelial junctions (IEJs). In unperturbed continuous endothelium, water, ions and small molecules are transported paracellularly by diffusion and convection. A significant amount of water (up to 40%) also crosses the endothelial cell barrier transcellularly through water-transporting membrane channels called aquaporins. A variety of stimuli can disrupt the organization of the IEJs, thereby opening gaps in the endothelial barrier. The formation of these intercellular gaps allows passage of fluid, ions, macromolecules (e.g., proteins) and other plasma constituents between the endothelial cells in an unrestricted manner. This paracellular-caused hyperpermeability produces edema and other adverse effects that can eventually result in damage to tissues and organs.

The transcellular pathway is responsible for the active transport of macromolecules, such as albumin and other plasma proteins, across the endothelial cells, a process referred to as “transcytosis.” The transport of macromolecules occurs in vesicles called caveolae. Almost all continuous endothelia have abundant caveolae, except for continuous endothelia located in brain and testes which have few caveolae. Transcytosis is a multi-step process that involves successive caveolae budding and fission from the plasmalemma and translocation across the cell, followed by docking and fusion with the opposite plasmalemma, where the caveolae release their contents by exocytosis into the interstitium. Transcytosis is selective and tightly regulated under normal physiological conditions.

There is a growing realization of the fundamental importance of the transcellular pathway. Transcytosis of plasma proteins, especially albumin which represents 65% of plasma protein, is of particular interest because of its ability to regulate the transvascular oncotic pressure gradient. As can be appreciated, then, increased transcytosis of albumin and other plasma proteins above basal levels will increase the tissue protein concentration of them which, in turn, will cause water to move across the endothelial barrier, thereby producing edema.

Low density lipoproteins (LDL) are also transported across endothelial cells by transcytosis. In hyperlipidemia, a significant increase in transcytosis of LDL has been detected as the initial event in atherogenesis. The LDL accumulates in the subendothelial space, trapped within the expanded basal lamina and extracellular matrix. The subendothelial lipoprotein accumulation in hyperlipidema is followed by a cascade of events resulting in atheromatous plaque formation. Advanced atherosclerotic lesions are reported to be occasionally accompanied by the opening of IEJs and massive uncontrolled passage of LDL and albumin.

Vascular complications are a hallmark of diabetes. At the level of large vessels, the disease appears to be expressed as an acceleration of an atherosclerotic process. With respect to microangiopathy, alterations in the microvasculature of the retina, renal glomerulus and nerves cause the greatest number of clinical complications, but a continuously increasing number of investigations show that diabetes also affects the microvasculature of other organs, such as the mesentery, skin, skeletal muscle, heart, brain and lung, causing additional clinical complications. In all of these vascular beds, changes in vascular permeability appear to represent a hallmark of the diabetic endothelial dysfunction.

In continuous endothelium, capillary hyperpermeability to plasma macromolecules in the early phase of diabetes is explained by an intensification of transendothelial vesicular transport (i.e., by increased transcytosis) and not by the destabilization of the IEJs. In addition, the endothelial cells of diabetics, including those of the brain, have been reported to contain an increased number of caveolae as compared to normals, and glycated proteins, particularly glycated albumin, are taken up by endothelial cells and transcytosed at substantially greater rates than their native forms. Further, increased transcytosis of macromolecules is a process that continues beyond the early phase of diabetes and appears to be a cause of edema in diabetic tissues and organs throughout the disease if left untreated. This edema, in turn, leads to tissue and organ damage. Similar increases in transcellular transport of macromolecules have been reported in hypertension.

Paracellular-caused hyperpermeability is also a factor in diabetes and the vascular complications of diabetes. The IEJs of the paracellular pathway include the adherens junctions (AJs) and tight junctions (TJs). Diabetes alters the content, phosphorylation and localization of certain proteins in both the AJs and TJs, thereby contributing to increased endothelial barrier permeability.

In support of the foregoing discussion and for further information, see Frank et al., Cell Tissue Res., 335:41-47 (2009), Simionescu et al., Cell Tissue Res., 335:27-40 (2009); van den Berg et al., J. Cyst. Fibros., 7(6): 515-519 (2008); Viazzi et al., Hypertens. Res., 31:873-879 (2008); Antonetti et al., Chapter 14, pages 340-342, in Diabetic Retinopathy (edited by Elia J. Duh, Humana Press, 2008), Felinski et al., Current Eye Research, 30:949-957 (2005), Pascariu et al., Journal of Histochemistry & Cytochemistry, 52(1):65-76 (2004); Bouchard et al., Diabetologia, 45:1017-1025 (2002); Arshi et al., Laboratory Investigation, 80(8):1171-1184 (2000); Vinores et al., Documenta Ophthalmologica, 97:217-228 (1999); Oomen et al., European Journal of Clinical Investigation, 29:1035-1040 (1999); Vinores et al., Pathol. Res. Pract., 194:497-505 (1998); Antonetti et al., Diabetes, 47:1953-1959 (1998), Popov et al., Acta Diabetol., 34:285-293 (1997); Yamaji et al., Circulation Research, 72:947-957 (1993); Vinores et al., Histochemical Journal, 25:648-663 (1993); Beals et al., Microvascular Research, 45:11-19 (1993); Caldwell et al., Investigative Ophthalmol. Visual Sci., 33 (5): 16101619 (1992).

Endothelial transport in fenestrated endothelium also occurs by transcytosis and the paracellular pathway. In addition, endothelial transport occurs by means of the fenestrae. Fenestrated endothelia show a remarkably high permeability to water and small hydrophilic solutes due to the presence of the fenestrae.

The fenestrae may or may not be covered by a diaphragm. The locations of endothelium with diaphragmed fenestrae include endocrine tissue (e.g., pancreatic islets and adrenal cortex), gastrointestinal mucosa and renal peritubular capillaries. The permeability to plasma proteins of fenestrated endothelium with diaphragmed fenestrae does not exceed that of continuous endothelium.

The locations of endothelium with nondiaphragmed fenestrae include the glomeruli of the kidneys. The glomerular fenestrated endothelium is covered by a glycocalyx that extends into the fenestrae (forming so-called “seive plugs”) and by a more loosely associated endothelial cell surface layer of glycoproteins. Mathematical analyses of functional permselectivity studies have concluded that the glomerular endothelial cell glycocalyx, including that present in the fenestrae, and its associated surface layer account for the retention of up to 95% of plasma proteins within the circulation.

Loss of fenestrae in the glomerular endothelium has been found to be associated with proteinuria in several diseases, including diabetic nephropathy, transplant glomerulopathy, pre-eclampsia, diabetes, renal failure, cyclosporine nephropathy, serum sickness nephritis and Thy-1 nephritis. Actin rearrangement and, in particular, depolymerization of stress fibers have been found to be important for the formation and maintenance of fenestrae.

In support of the foregoing discussion of fenestrated endothelia and for additional information, see Satchell et al., Am. J. Physiol. Renal Physiol., 296:F947-F956 (2009); Haral ds son et al., Curr. Opin. Nephrol. Hypertens., 18:331-335 (2009); Ichimura et al., J. Am. Soc. Nephrol., 19:1463-1471 (2008); Ballermann, Nephron Physiol., 106:19-25 (2007); Toyoda et al., Diabetes, 56:2155-2160 (2007); Stan, “Endothelial Structures Involved In Vascular Permeability,” pages 679-688, Endothelial Biomedicine (ed. Aird, Cambridge University Press, Cambridge, 2007); Simionescu and Antohe, “Functional Ultrastructure of the Vascular Endothelium: Changes in Various Pathologies,” pages 42-69, The Vascular Endothelium I (eds. Moncada and Higgs, Springer-Verlag, Berlin, 2006).

Endothelial transport in sinusoidal endothelium occurs by transcytosis and through the intercellular gaps (interendothelial slits) and intracellular gaps (fenestrae). Treatment of sinusoidal endothelium with actin filament-disrupting drugs can induce a substantial and rapid increase in the number of gaps, indicating regulation of the porosity of the endothelial lining by the actin cytoskeleton. Other cytoskeleton altering drugs have been reported to change the diameters of fenestrae. Therefore, the fenestrae-associated cytoskeleton probably controls the important function of endothelial filtration in sinusodial endotheluium. In liver, defenestration (loss of fenestrae), which causes a reduction in permeability of the endothelium, has been associated with the pathogenesis of several diseases and conditions, including aging, atherogenesis, atherosclerosis, cirrhosis, fibrosis, liver failure and primary and metastatic liver cancers. In support of the foregoing and for additional information, see Yokomori, Med. Mol. Morphol., 41:1-4 (2008); Stan, “Endothelial Structures Involved In Vascular Permeability,” pages 679-688, Endothelial Biomedicine (ed. Aird, Cambridge University Press, Cambridge, 2007); DeLeve, “The Hepatic Sinusoidal Endothelial Cell,” pages 1226-1238, Endothelial Biomedicine (ed. Aird, Cambridge University Press, Cambridge, 2007); Pries and Kuebler, “Normal Endothelium,” pages 1-40, The Vascular Endothelium I (eds. Moncada and Higgs, Springer-Verlag, Berlin, 2006); Simionescu and Antohe, “Functional Ultrastructure of the Vascular Endothelium: Changes in Various Pathologies,” pages 42-69, The Vascular Endothelium I (eds. Moncada and Higgs, Springer-Verlag, Berlin, 2006); Braet and Wisse, Comparative Hepatology, 1:1-17 (2002); Kanai et al., Anat. Rec., 244:175-181 (1996); Kempka et al., Exp. Cell Res., 176:38-48 (1988); Kishimoto et al., Am. J. Anat., 178:241-249 (1987).

The invention provides a method of inhibiting vascular hyperpermeability present in any tissue or organ containing or surrounded by continuous endothelium. As noted above, continuous endothelium is present in, or around, the brain (blood brain barrier), diaphragm, duodenal musculature, fat, heart, some areas of the kidneys (papillary microvasculature, descending vasa recta), large blood vessels, lungs, mesentery, nerves, retina (blood retinal barrier), skeletal muscle, skin, testis, umbilical vein and other tissues and organs of the body. Preferably, the continuous endothelium is that found in or around the brain, heart, lungs, nerves or retina.

The invention also provides a method of inhibiting vascular hyperpermeability present in any tissue or organ containing or surrounded by fenestrated endothelium. As noted above, fenestrated endothelium is present in, or around, the kidney (glomeruli, peritubular capillaries and ascending vasa recta), pancreas, adrenal glands, endocrine glands and intestine. Preferably, the fenestrated endothelium is that found in the kidneys, especially that found in the glomeruli of the kidneys.

The data presented in the Examples below, provides evidence that the combination of LMWFHSA and a p38 MAPK inhibitor reduces endothelial permeability by a microtubule mediated mechanism by an unexpected amount as compared to LMWFHSA, thus the invention provides for a method of inhibiting vascular hyperpermeability in an animal in need thereof comprising administering to the animal an effective amount of a pharmaceutical composition comprising LMWFHSA and one or more p38 MAPK inhibitors. In still another aspect, the invention provides for a pharmaceutical composition comprising LMWFHSA with one or more p38 MAPK inhibitors for the treatment of vascular disorders and/or for the inhibition of vascular hyperpermeability. In one aspect, the p38 MAPK inhibitors are selected from the group consisting of SB 203580, SB 203580 hydrochloride, SB 202190, SB 239063, SB 706504, AL 8697, AMG 548, CMPD-1, DBM 1285 dihydrochloride, EO 1428, JX 401, ML 3403, RWJ 67657, SCIO 469 hydrochloride, SKF 86002 dihydrochloride, SX 011, TA 01, TA 02, TAK 715, VX 702, VX 745, p38 MAPK Inhibitor TOCRISET™, and combinations thereof.

Further, any disease or condition mediated by vascular hyperpermeability can be treated by the method of the invention to inhibit the vascular hyperpermeability. Such diseases and conditions include diabetes, hypertension, atherosclerosis and ocular diseases.

In particular, the vascular complications of diabetes, including those of the brain, heart, kidneys, lung, mesentery, nerves, retina, skeletal muscle, skin and other tissues and organs containing continuous or fenestrated endothelium, can be treated by the present invention. These vascular complications include edema, accumulation of LDL in the subendothelial space, accelerated atherosclerosis, and the following: brain (accelerated aging of vessel walls), heart (myocardial edema, myocardial fibrosis, diastolic dysfunction, diabetic cardiomyopathy), kidneys (diabetic nephropathy), lung (retardation of lung development in the fetuses of diabetic mothers, alterations of several pulmonary physiological parameters and increased susceptibility to infections), mesentery (vascular hyperplasy), nerves (diabetic neuropathy), retina (macular edema and diabetic retinopathy) and skin (redness, discoloration, dryness and ulcerations). Vascular hyperpermeability in both Type 1 (autoimmune) and Type 2 (non-insulin-dependent) diabetes can be inhibited by the method of the invention. Type 2 is the most common type of diabetes, affecting 90-95% of diabetics, and its treatment, especially the treatment of those with early signs of, or a predisposition to develop, Type 2 diabetes (see below), should be particularly beneficial.

Diabetic retinopathy is a leading cause of blindness that affects approximately 25% of the estimated 21 million Americans with diabetes. Although its incidence and progression can be reduced by intensive glycemic and blood pressure control, nearly all patients with type 1 diabetes mellitus and over 60% of those with type 2 diabetes mellitus eventually develop diabetic retinopathy. There are two stages of diabetic retinopathy. The first, non-proliferative retinopathy, is the earlier stage of the disease and is characterized by increased vascular permeability, microaneurysms, edema and eventually vessel closures. Neovascularization is not a component of the nonproliferative phase. Most visual loss during this stage is due to the fluid accumulating in the macula, the central area of the retina. This accumulation of fluid is called macular edema and can cause temporary or permanent decreased vision. The second stage of diabetic retinopathy is called proliferative retinopathy and is characterized by abnormal new vessel formation. Unfortunately, this abnormal neovascularization can be very damaging because it can cause bleeding in the eye, retinal scar tissue, diabetic retinal detachments or glaucoma, any of which can cause decreased vision or blindness. Macular edema can also occur in the proliferative phase.

Diabetic neuropathy is a common serious complication of diabetes. There are four main types of diabetic neuropathy: peripheral neuropathy, autonomic neuropathy, radiculoplexus neuropathy and mononeuropathy. The signs and symptoms of peripheral neuropathy, the most common type of diabetic neuropathy, include numbness or reduced ability to feel pain or changes in temperature (especially in the feet and toes), a tingling or burning feeling, sharp pain, pain when walking, extreme sensitivity to the lightest touch, muscle weakness, difficulty walking, and serious foot problems (such as ulcers, infections, deformities and bone and joint pain). Autonomic neuropathy affects the autonomic nervous system that controls the heart, bladder, lungs, stomach, intestines, sex organs and eyes, and problems in any of these areas can occur. Radiculoplexus neuropathy (also called diabetic amyotrophy, femoral neuropathy or proximal neuropathy) usually affects nerves in the hips, shoulders or abdomen, usually on one side of the body. Mononeuropathy means damage to just one nerve, typically in an arm, leg or the face. Common complications of diabetic neuropathy include loss of limbs (e.g., toes, feet or legs), charcot joints, urinary tract infections, urinary incontinence, hypoglycemia unawareness (may even be fatal), low blood pressure, digestive problems (e.g., constipation, diarrhea, nausea and vomiting), sexual dysfunction (e.g., erectile dysfunction), and increased or decreased sweating. As can be seen, symptoms can range from mild to painful, disabling and even fatal.

Diabetic nephropathy is the most common cause of end-stage renal disease in the United States. It is a vascular complication of diabetes that affects the glomerular capillaries of the kidney and reduces the kidney's filtration ability. Nephropathy is first indicated by the appearance of hyperfiltration and then microalbuminuria. Heavy proteinuria and a progressive decline in renal function precede end-stage renal disease. Typically, before any signs of nephropathy appear, retinopathy has usually been diagnosed. Renal transplant is usually recommended to patients with end-stage renal disease due to diabetes. Survival rate at 5 years for patients receiving a transplant is about 60% compared with only 2% for those on dialysis.

Hypertension typically develops over many years, and it affects nearly everyone eventually. Uncontrolled hypertension increases the risk of serious health problems, including heart attack, congestive heart failure, stroke, peripheral artery disease, kidney failure, aneurysms, eye damage, and problems with memory or understanding.

Atherosclerosis also develops gradually. Atherosclerosis can affect the coronary arteries, the carotid artery, the peripheral arteries or the microvasculature, and complications of atherosclerosis include coronary artery disease (which can cause angina or a heart attack), coronary microvascular disease, carotid artery disease (which can cause a transient ischemic attack or stroke), peripheral artery disease (which can cause loss of sensitivity to heat and cold or even tissue death), and aneurysms.

Additional diseases and conditions that can be treated according to the invention include acute lung injury, age-related macular degeneration, choroidal edema, choroiditis, coronary microvascular disease, cerebral microvascular disease, Eals disease, edema caused by injury (e.g., trauma or burns), edema associated with hypertension, glomerular vascular leakage, hemorrhagic shock, Irvine Gass Syndrome, edema caused by ischemia, macular edema (e.g., caused by vascular occlusions, post-intraocular surgery (e.g., cataract surgery), uveitis or retinitis pigmentosa, in addition to that caused by diabetes), nephritis (e.g., glomerulonephritis, serum sickness nephritis and Thy-1 nephritis), nephropathies, nephrotic edema, nephrotic syndrome, neuropathies, organ failure due to tissue edema (e.g., in sepsis or due to trauma), pre-eclampsia, pulmonary edema, pulmonary hypertension, renal failure, retinal edema, retinal hemorrhage, retinal vein occlusions (e.g., branch or central vein occlusions), retinitis, retinopathies (e.g., artherosclerotic retinopathy, hypertensive retinopathy, radiation retinopathy, sickle cell retinopathy and retinopathy of prematurity, in addition to diabetic retinopathy), silent cerebral infarction, systemic inflammatory response syndromes (SIRS), transplant glomerulopathy, uveitis, vascular leakage syndrome, vitreous hemorrhage and Von Hipple Lindau disease. In addition, certain drugs, including those used to treat multiple sclerosis, are known to cause vascular hyperpermeability, and a diketopiperazine, a prodrug of a diketopiperazine or a pharmaceutically-acceptable salt of either one of them, can be used to reduce this unwanted side effect when using these drugs.

“Treat,” “treating” or “treatment” is used herein to mean to reduce (wholly or partially) the symptoms, duration or severity of a disease or condition.

Recent evidence indicates that transcytosis-caused hyperpermeability is the first step of a process that ultimately leads to tissue and organ damage in many diseases and conditions. Accordingly, the present invention provides a means of early intervention in these diseases and conditions which can reduce, delay or even potentially prevent the tissue and organ damage seen in them. For instance, an animal can be treated immediately upon diagnosis of one of the diseases or conditions treatable according to the invention (those diseases and conditions described above).

Alternatively, preferred is the treatment of animals who have early signs of, or a predisposition to develop, such a disease or condition prior to the existence of symptoms. Early signs of, and risk factors for, diabetes, hypertension and atherosclerosis are well known, and treatment of an animal exhibiting these early signs or risk factors can be started prior to the presence of symptoms of the disease or condition (i.e., prophylactically).

For instance, treatment of a patient who is diagnosed with diabetes can be started immediately upon diagnosis. In particular, diabetics should preferably be treated with a combination of LMWFHSA and a p38 MAPK inhibitor prior to any symptoms of a vascular complication being present, although this is not usually possible, since most diabetics show such symptoms when they are diagnosed (see below). Alternatively, diabetics should be treated while nonproliferative diabetic retinopathy is mild (i.e., mild levels of microaneurysms and intraretinal hemorrhage). See Diabetic Retinopathy, page 9 (Ed. Elia Duh, M.D., Human Press, 2008). Such early treatment will provide the best chance of preventing macular edema and progression of the retinopathy to proliferative diabetic retinopathy. Also, the presence of diabetic retinopathy is considered a sign that other microvascular complications of diabetes exist or will develop (see Id., pages 474-477), and early treatment may also prevent or reduce these additional complications. Of course, more advanced diseases and conditions that are vascular complications of diabetes can also be treated with beneficial results.

However, as noted above, vascular complications are often already present by the time diabetes is diagnosed. Accordingly, it is preferable to prophylactically treat a patient who has early signs of, or a predisposition to develop, diabetes. The early signs and risk factors of Type 2 diabetes include fasting glucose that is high, but not high enough to be classified as diabetes (“prediabetes”), hyperinsulinemia, hypertension, dyslipidemia (high cholesterol, high triglycerides, high low-density lipoprotein, and/or low level of high-density lipoprotein), obesity (body mass index above 25), inactivity, over 45 years of age, inadequate sleep, family history of diabetes, minority race, history of gestational diabetes, history of polycystic ovary syndrome and diagnosis of metabolic syndrome. Accordingly, patients with early signs of, or a predisposition to develop, Type 2 diabetes can readily be treated prophylactically.

Similarly, treatment of a patient who is diagnosed with hypertension can be started immediately upon diagnosis. Hypertension typically does not cause any symptoms, but prophylactic treatment can be started in a patient who has a predisposition to develop hypertension. Risk factors for hypertension include age, race (hypertension is more common blacks), family history (hypertension runs in families), overweight or obesity, lack of activity, smoking tobacco, too much salt in the diet, too little potassium in the diet, too little vitamin D in the diet, drinking too much alcohol, high levels of stress, certain chronic conditions (e.g., high cholesterol, diabetes, kidney disease and sleep apnea) and use of certain drugs (e.g., oral contraceptives, amphetamines, diet pills, and some cold and allergy medications).

Treatment of a patient who is diagnosed with atherosclerosis can be started immediately upon diagnosis. However, it is preferable to prophylactically treat a patient who has early signs of, or a predisposition to develop, atherosclerosis. Early signs and risk factors for atherosclerosis include age, a family history of aneurysm or early heart disease, hypertension, high cholesterol, high triglycerides, insulin resistance, diabetes, obesity, smoking, lack of physical activity, unhealthy diet, and high level of C-reactive protein.

The method of the invention for inhibiting vascular hyperpermeability comprises administering an effective amount of an active ingredient, wherein the active ingredient comprises LMWFHSA and one or more p38 MAPK inhibitors, to an animal in need thereof to inhibit the vascular hyperpermeability.

The LMWFHSA composition and/or products of the present invention can be prepared from solutions, including from the commercially-available pharmaceutical compositions comprising albumin, such as human serum albumin, by well-known methods, such as ultrafiltration, chromatography (size-exclusion chromatography, Centricon filtration, affinity chromatography (e.g., using a column of beads having attached thereto an antibody or antibodies directed to the desired diketopiperazine(s) or an antibody or antibodies directed to the truncated protein or peptide), anion exchange or cation exchange), sucrose gradient centrifugation, chromatography, salt precipitation, or sonication, that will remove some or all of the albumin in the solution. The resultant LMWFHSA-containing composition and/or product can be used and incorporated into pharmaceutical compositions as described above.

Using an ultrafilration separation method, a human serum albumin composition can be passed over an ultrafiltration membrane having a molecular weight cut-off that retains the albumin while allowing lower molecular weight components to pass into the resulting filtrate or fraction. This filtrate may comprise components having molecular weights less than about 50 kDA, less than about 40 kDa, less than 30 kDa, less than about 20 kDa, less than about 10 kDa, less than about 5 kDa, less than about 3 kDa. Preferably, the filtrate comprises components having molecular weights less than about 5 Da (also referred to as “<5000 MW” or LMWF5A). This <5000 MW or LMWF5A fraction or filtrate contains aspartyl-alanyl diketopiperazine (“DA-DKP”) which is formed after the dipeptide aspartate-alanine is cleaved from albumin and subsequently cyclized into the diketopiperazine.

The physiologically-acceptable salts of LMWFHSA of the invention may also be used in the practice of the invention. Physiologically-acceptable salts include conventional non-toxic salts, such as salts derived from inorganic acids (such as hydrochloric, hydrobromic, sulfuric, phosphoric, nitric, and the like), organic acids (such as acetic, propionic, succinic, glycolic, stearic, lactic, malic, tartaric, citric, glutamic, aspartic, benzoic, salicylic, oxalic, ascorbic acid, and the like) or bases (such as the hydroxide, carbonate or bicarbonate of a pharmaceutically-acceptable metal cation or organic cations derived from N,N-dibenzylethylenediamine, D-glucosamine, or ethylenediamine). The salts are prepared in a conventional manner, e.g., by neutralizing the free base form of the compound with an acid.

As noted above, LMWFHSA and one or more p38 MAPK inhibitors can be used to inhibit vascular hyperpermeability and to treat a disease or condition mediated by vascular hyperpermeability. To do so, the LMWFHSAP and the p38 MAPK inhibitors are administered to an animal in need of treatment. Preferably, the animal is a mammal, such as a rabbit, goat, dog, cat, horse or human. Most preferably, the animal is a human.

The LMWFHSA and the one or more p38 MAPK inhibitors as used in the present invention are used as active ingredients. “Active ingredient” is used herein to mean a compound having therapeutic, pharmaceutical or pharmacological activity, and particularly, the therapeutic, pharmaceutical or pharmacological activity described herein. The LMWFHSA is not used in the present invention as a carrier or as part of a carrier system of a pharmaceutical composition as described in, e.g., U.S. Pat. Nos. 5,976,569, 6,099,856, 7,276,534 and PCT applications WO 96/10396, WO 2006/023943, WO 2007/098500, WO 2007/121411 and WO 2010/102148.

Effective dosage forms, modes of administration and dosage amounts for the compositions of the invention may be determined empirically using the guidance provided herein. It is understood by those skilled in the art that the dosage amount will vary with the particular disease or condition to be treated, the severity of the disease or condition, the route(s) of administration, the duration of the treatment, the identity of any other drugs being administered to the animal, the age, size and species of the animal, and like factors known in the medical and veterinary arts. In general, a suitable dose of a composition of the present invention will be that amount of the composition which is the lowest dose effective to produce a therapeutic effect. However, the dosage will be determined by an attending physician or veterinarian within the scope of sound medical judgment. If desired, effective doses may be administered as two, three, four, five, six or more sub-doses, administered separately at appropriate intervals throughout the day or other time period. Administration of the composition should be continued until an acceptable response is achieved.

The LMWFHSA composition of the invention can be administered concurrently, sequentially, or intermittently with one or more p38 MAPK inhibitors.

In one aspect of the invention, one or more p38 MAPK inhibitors are administered sequentially with the LMWFHSA composition. In another embodiment, one or more p38 MAPK inhibitors are administered before the LMWFHSA composition is administered. In another embodiment, one or more additional p38 MAPK inhibitors are administered after the LMWFHSA composition is administered. In one embodiment, one or more additional p38 MAPK inhibitors are administered in alternating doses with the LMWFHSA composition, or in a protocol in which the LMWFHSA composition is administered at prescribed intervals in between or with one or more consecutive doses of the one or more p38 MAPK inhibitors, or vice versa. In one embodiment, the LMWFHSA composition is administered in one or more doses over a period of time prior to commencing the administration of the one or more p38 MAPK inhibitors. In other words, the LMWFHSA composition is administered as a monotherapy for a period of time, and then the p38 MAPK inhibitor administration is added, either concurrently with new doses of the LMWFHSA, or in an alternating fashion with LMWFHSA. Alternatively, the one or more p38 MAPK inhibitors may be administered for a period of time prior to beginning administration of the LMWFHSA composition.

In one aspect of the invention, when a treatment course of the one or more p38 MAPK inhibitors begins, additional doses of the LMWFHSA composition are administered over the same period of time, or for at least a portion of that time, and may continue to be administered once the course of the p38 MAPK inhibitor has ended. However, the dosing schedule for the LMWFHSA over the entire period can be, different than that for the one or more p38 MAPK inhibitors. For example, the LMWFHSA composition may be administered on the same days or at least 1-4 days after the last given (most recent) dose of the one or more p38 MAPK inhibitors (or any suitable number of days after the last dose), and may be administered daily, weekly, biweekly, monthly, bimonthly, or every 3-6 months, or at longer intervals as determined by the physician.

In aspects of the invention, the LMWFHSA composition and the one or more p38 MAPK inhibitors can be administered together (concurrently). As used herein, concurrent use does not necessarily mean that all doses of all compounds are administered on the same day at the same time. Rather, concurrent use means that each of the components (e.g., LMWFHSA and the one or more p38 MAPK inhibitors) are started at approximately the same period (within hours) and are administered over the same general period of time, noting that each component may have a different dosing schedule. In addition, before or after the concurrent administration period, any one of the LMWFHSA or p38 MAPK inhibitors compositions be administered without the other.

The invention also provides a method of modulating the cytoskeleton of endothelial cells in an animal. Modulation of the cytoskeleton can reduce vascular hyperpermeability and increase vascular hypopermeability (i.e., permeability below basal levels), thereby returning the endothelium to homeostasis. Accordingly, those diseases and conditions mediated by vascular hyperpermeability can be treated (see above) and those diseases and conditions mediated by vascular hypopermeability can also be treated. The latter type of diseases and conditions include aging liver, atherogenesis, atherosclerosis, cirrhosis, fibrosis of the liver, liver failure and primary and metastatic liver cancers.

The method of modulating the cytoskeleton of endothelial cells comprises administering an effective amount of LMWFHSA and one or more p38 MAPK inhibitors to an animal. The diketopiperazines are the same as those described above for inhibiting vascular hyperpermeability, and “animal” has the same meaning as set forth above.

Effective dosage forms, modes of administration and dosage amounts for the compositions of the invention for modulating the cytoskeleton may be determined empirically using the guidance provided herein. It is understood by those skilled in the art that the dosage amount will vary with the particular disease or condition to be treated, the severity of the disease or condition, the route(s) of administration, the duration of the treatment, the identity of any other drugs being administered to the animal, the age, size and species of the animal, and like factors known in the medical and veterinary arts. In general, a suitable daily dose of a compound of the present invention will be that amount of the compound which is the lowest dose effective to produce a therapeutic effect. However, the daily dosage will be determined by an attending physician or veterinarian within the scope of sound medical judgment. If desired, the effective daily dose may be administered as two, three, four, five, six or more sub-doses, administered separately at appropriate intervals throughout the day. Administration of the compound should be continued until an acceptable response is achieved.

The LMWFHSA and the one or more p38 MAPK inhibitor components of the composition of the present invention may be administered to an animal patient for therapy by any suitable route of administration, including orally, nasally, parenterally (e.g., intravenously, intraperitoneally, subcutaneously or intramuscularly), transdermally, intraocularly and topically (including buccally and sublingually) and the routes of administration of the LMWFHSA may be the same or different as the routes of administration of the one or more p38 MAPK inhibitor. For example, the route of administration of the LMWFHSA can be topically by eye drops, whereas the one or more p38 MAPK inhibitor can be administered concurrently or sequentially by an oral administration route. The preferred routes of administration of the LMWFHSA for treatment of diseases and conditions of the eye are orally, intraocularly and topically. Most preferred is topically. The preferred routes of administration of the LMWFHSA for treatment of diseases and conditions of the brain are orally and parenterally. Most preferred is orally.

While it is possible for a compound of the present invention to be administered alone, it is preferable to administer the compound as a pharmaceutical formulation (composition). The pharmaceutical compositions of the invention comprise a compound or compounds of the invention as an active ingredient in admixture with one or more pharmaceutically-acceptable carriers and, optionally, with one or more other compounds, drugs or other materials. Each carrier must be “acceptable” in the sense of being compatible with the other ingredients of the formulation and not injurious to the animal. Pharmaceutically-acceptable carriers are well known in the art. Regardless of the route of administration selected, the compounds of the present invention are formulated into pharmaceutically-acceptable dosage forms by conventional methods known to those of skill in the art. See, e.g., Remington's Pharmaceutical Sciences.

Formulations of the invention suitable for oral administration may be in the form of capsules, cachets, pills, tablets, powders, granules or as a solution or a suspension in an aqueous or non-aqueous liquid, or an oil-in-water or water-in-oil liquid emulsions, or as an elixir or syrup, or as pastilles (using an inert base, such as gelatin and glycerin, or sucrose and acacia), and the like, each containing a predetermined amount of a compound or compounds of the present invention as an active ingredient. A compound or compounds of the present invention may also be administered as bolus, electuary or paste.

In solid dosage forms of the invention for oral administration (capsules, tablets, pills, dragees, powders, granules and the like), the active ingredient (i.e., a diketopiperazine of formula I, a prodrug of a diketopiperazine of formula I, a pharmaceutically-acceptable salt of either one of them, or combinations of the foregoing) is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monosterate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may be employed as fillers in soft and hard-filled gelatin capsules using such excipients as lactose or milk sugars, as well as high molecular weight polyethylene glycols and the like.

A tablet may be made by compression or molding optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter. These compositions may also optionally contain opacifying agents and may be of a composition that they release the active ingredient only, or preferentially, in a certain portion of the gastrointestinal tract, optionally, in a delayed manner. Examples of embedding compositions which can be used include polymeric substances and waxes. The active ingredient can also be in microencapsulated form.

Liquid dosage forms for oral administration of the compounds of the invention include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active ingredient, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The invention also provides pharmaceutical products suitable for treatment of the eye. Such pharmaceutical products include pharmaceutical compositions, devices and implants (which may be compositions or devices).

Pharmaceutical formulations (compositions) for intraocular injection of a compound or compounds of the invention into the eyeball include solutions, emulsions, suspensions, particles, capsules, microspheres, liposomes, matrices, etc. See, e.g., U.S. Pat. No. 6,060,463, U.S. Patent Application Publication No. 2005/0101582, and PCT application WO 2004/043480, the complete disclosures of which are incorporated herein by reference. For instance, a pharmaceutical formulation for intraocular injection may comprise one or more compounds of the invention in combination with one or more pharmaceutically-acceptable sterile isotonic aqueous or non-aqueous solutions, suspensions or emulsions, which may contain antioxidants, buffers, suspending agents, thickening agents or viscosity-enhancing agents (such as a hyaluronic acid polymer). Examples of suitable aqueous and nonaqueous carriers include water, saline (preferably 0.9%), dextrose in water (preferably 5%), buffers, dimethylsulfoxide, alcohols and polyols (such as glycerol, propylene glycol, polyethylene glycol, and the like). These compositions may also contain adjuvants such as wetting agents and emulsifying agents and dispersing agents. In addition, prolonged absorption of the injectable pharmaceutical form may be brought about by the inclusion of agents which delay absorption such as polymers and gelatin. Injectable depot forms can be made by incorporating the drug into microcapsules or microspheres made of biodegradable polymers such as polylactide-polyglycolide. Examples of other biodegradable polymers include poly(orthoesters), poly(glycolic) acid, poly(lactic) acid, polycaprolactone and poly(anhydrides). Depot injectable formulations are also prepared by entrapping the drug in liposomes (composed of the usual ingredients, such as dipalmitoyl phosphatidylcholine) or microemulsions which are compatible with eye tissue. Depending on the ratio of drug to polymer or lipid, the nature of the particular polymer or lipid components, the type of liposome employed, and whether the microcapsules or microspheres are coated or uncoated, the rate of drug release from microcapsules, microspheres and liposomes can be controlled.

The compounds of the invention can also be administered surgically as an ocular implant. For instance, a reservoir container having a diffusible wall of polyvinyl alcohol or polyvinyl acetate and containing a compound or compounds of the invention can be implanted in or on the sclera. As another example, a compound or compounds of the invention can be incorporated into a polymeric matrix made of a polymer, such as polycaprolactone, poly(glycolic) acid, poly(lactic) acid, poly(anhydride), or a lipid, such as sebacic acid, and may be implanted on the sclera or in the eye. This is usually accomplished with the animal receiving a topical or local anesthetic and using a small incision made behind the cornea. The matrix is then inserted through the incision and sutured to the sclera.

The compounds of the invention can also be administered topically to the eye, and a preferred embodiment of the invention is a topical pharmaceutical composition suitable for application to the eye. Topical pharmaceutical compositions suitable for application to the eye include solutions, suspensions, dispersions, drops, gels, hydrogels and ointments. See, e.g., U.S. Pat. No. 5,407,926 and PCT applications WO 2004/058289, WO 01/30337 and WO 01/68053, the complete disclosures of all of which are incorporated herein by reference.

Topical formulations suitable for application to the eye comprise one or more compounds of the invention in an aqueous or nonaqueous base. The topical formulations can also include absorption enhancers, permeation enhancers, thickening agents, viscosity enhancers, agents for adjusting and/or maintaining the pH, agents to adjust the osmotic pressure, preservatives, surfactants, buffers, salts (preferably sodium chloride), suspending agents, dispersing agents, solubilizing agents, stabilizers and/or tonicity agents. Topical formulations suitable for application to the eye will preferably comprise an absorption or permeation enhancer to promote absorption or permeation of the compound or compounds of the invention into the eye and/or a thickening agent or viscosity enhancer that is capable of increasing the residence time of a compound or compounds of the invention in the eye. See PCT applications WO 2004/058289, WO 01/30337 and WO 01/68053. Exemplary absorption/permeation enhancers include methysulfonylmethane, alone or in combination with dimethylsulfoxide, carboxylic acids and surfactants. Exemplary thickening agents and viscosity enhancers include dextrans, polyethylene glycols, polyvinylpyrrolidone, polysaccharide gels, Gelrite®, cellulosic polymers (such as hydroxypropyl methylcellulose), carboxyl-containing polymers (such as polymers or copolymers of acrylic acid), polyvinyl alcohol and hyaluronic acid or a salt thereof.

Liquid dosage forms (e.g., solutions, suspensions, dispersions and drops) suitable for treatment of the eye can be prepared, for example, by dissolving, dispersing, suspending, etc. a compound or compounds of the invention in a vehicle, such as, for example, water, saline, aqueous dextrose, glycerol, ethanol and the like, to form a solution, dispersion or suspension. If desired, the pharmaceutical formulation may also contain minor amounts of non-toxic auxillary substances, such as wetting or emulsifying agents, pH buffering agents and the like, for example sodium acetate, sorbitan monolaurate, triethanolamine sodium acetate, triethanolamine oleate, etc.

Aqueous solutions and suspensions suitable for treatment of the eye can include, in addition to a compound or compounds of the invention, preservatives, surfactants, buffers, salts (preferably sodium chloride), tonicity agents and water. If suspensions are used, the particle sizes should be less than 10 μm to minimize eye irritation. If solutions or suspensions are used, the amount delivered to the eye should not exceed 50 μl to avoid excessive spillage from the eye.

Colloidal suspensions suitable for treatment of the eye are generally formed from microparticles (i.e., microspheres, nanospheres, microcapsules or nanocapsules, where microspheres and nanospheres are generally monolithic particles of a polymer matrix in which the formulation is trapped, adsorbed, or otherwise contained, while with microcapsules and nanocapsules the formulation is actually encapsulated). The upper limit for the size of these microparticles is about 5μ to about 10μ.

Ophthalmic ointments suitable for treatment of the eye include a compound or compounds of the invention in an appropriate base, such as mineral oil, liquid lanolin, white petrolatum, a combination of two or all three of the foregoing, or polyethylene-mineral oil gel. A preservative may optionally be included.

Ophthalmic gels suitable for treatment of the eye include a compound or compounds of the invention suspended in a hydrophilic base, such as Carpobol-940 or a combination of ethanol, water and propylene glycol (e.g., in a ratio of 40:40:20). A gelling agent, such as hydroxylethylcellulose, hydroxypropylcellulose, hydroxypropylmethylcellulose or ammoniated glycyrrhizinate, is used. A preservative and/or a tonicity agent may optionally be included.

Hydrogels suitable for treatment of the eye are formed by incorporation of a swellable, gel-forming polymer, such as those listed above as thickening agents or viscosity enhancers, except that a formulation referred to in the art as a “hydrogel” typically has a higher viscosity than a formulation referred to as a “thickened” solution or suspension. In contrast to such preformed hydrogels, a formulation may also be prepared so to form a hydrogel in situ following application to the eye. Such gels are liquid at room temperature but gel at higher temperatures (and thus are termed “thermoreversible” hydrogels), such as when placed in contact with body fluids. Biocompatible polymers that impart this property include acrylic acid polymers and copolymers, N-isopropylacrylamide derivatives and ABA block copolymers of ethylene oxide and propylene oxide (conventionally referred to as “poloxamers” and available under the PLURONIC® tradename from BASF-Wayndotte).

Preferred dispersions are liposomal, in which case the formulation is enclosed within liposomes (microscopic vesicles composed of alternating aqueous compartments and lipid bilayers).

Eye drops can be formulated with an aqueous or nonaqueous base also comprising one or more dispersing agents, solubilizing agents or suspending agents. Drops can be delivered by means of a simple eye dropper-capped bottle or by means of a plastic bottle adapted to deliver liquid contents dropwise by means of a specially shaped closure.

The compounds of the invention can also be applied topically by means of drug-impregnated solid carrier that is inserted into the eye. Drug release is generally effected by dissolution or bioerosion of the polymer, osmosis, or combinations thereof. Several matrix-type delivery systems can be used. Such systems include hydrophilic soft contact lenses impregnated or soaked with the desired compound of the invention, as well as biodegradable or soluble devices that need not be removed after placement in the eye. These soluble ocular inserts can be composed of any degradable substance that can be tolerated by the eye and that is compatible with the compound of the invention that is to be administered. Such substances include, but are not limited to, poly(vinyl alcohol), polymers and copolymers of polyacrylamide, ethylacrylate and vinylpyrrolidone, as well as cross-linked polypeptides or polysaccharides, such as chitin.

Dosage forms for the other types of topical administration (i.e., not to the eye) or for transdermal administration of compounds of the invention include powders, sprays, ointments, pastes, creams, lotions, gels, solutions, patches, drops and inhalants. The active ingredient may be mixed under sterile conditions with a pharmaceutically-acceptable carrier, and with any buffers, or propellants which may be required. The ointments, pastes, creams and gels may contain, in addition to the active ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to the active ingredient, excipients such as lactose, talc, silicic acid, aluminium hydroxide, calcium silicates and polyamide powder or mixtures of these substances. Sprays can additionally contain customary propellants such as chlorofluorohydrocarbons and volatile unsubstituted hydrocarbons, such as butane and propane. Transdermal patches have the added advantage of providing controlled delivery of compounds of the invention to the body. Such dosage forms can be made by dissolving, dispersing or otherwise incorporating one or more compounds of the invention in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel. A drug-impregnated solid carrier (e.g., a dressing) can also be used for topical administration.

Pharmaceutical formulations include those suitable for administration by inhalation or insufflation or for nasal administration. For administration to the upper (nasal) or lower respiratory tract by inhalation, the compounds of the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of one or more compounds of the invention and a suitable powder base, such as lactose or starch. The powder composition may be presented in unit dosage form in, for example, capsules or cartridges, or, e.g., gelatin or blister packs from which the powder may be administered with the aid of an inhalator, insufflator or a metered-dose inhaler.

For intranasal administration, compounds of the invention may be administered by means of nose drops or a liquid spray, such as by means of a plastic bottle atomizer or metered-dose inhaler. Liquid sprays are conveniently delivered from pressurized packs. Typical of atomizers are the Mistometer (Wintrop) and Medihaler (Riker).

Nose drops may be formulated with an aqueous or nonaqueous base also comprising one or more dispersing agents, solubilizing agents or suspending agents. Drops can be delivered by means of a simple eye dropper-capped bottle or by means of a plastic bottle adapted to deliver liquid contents dropwise by means of a specially shaped closure.

Pharmaceutical compositions of this invention suitable for parenteral administrations comprise one or more compounds of the invention in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents. Also, drug-coated stents may be used.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents which delay absorption such as aluminium monosterate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug is accomplished by dissolving or suspending the drug in an oil vehicle.

Injectable depot forms are made by forming microencapsule matrices of the drug in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

As disclosed in the Examples presented herein, evidence is provided that a LMWFHSA composition of the present invention (also referred to herein, for certain embodiments, as “LMWF5A”) reduces endothelial permeability in vitro. Permeability assays demonstrate that treatment of HREC with LMWFHSA decreases the passage of macromolecular solutes as well as provides protection against the paracellular route that accompanies medium exhaustion in extended culture. These changes were concurrent with an increase in the acetylation of α-tubulin implying that microtubule dynamics are involved in the said activity of LMWFHSA.

One of the primary functions of microtubules is to provide the scaffolding necessary for intracellular trafficking. Dynein and kinesin molecular motors track along these dynamic polymers as they explore the cytoplasm to deliver cargo (Etienne-Manneville S. From signalling pathways to microtubule dynamics: the key players. Curr Opin Cell Biol 2010; 22:104-111). It has also been well documented that the acetylation of tubulin controls both kinesin affinity and the directionality of transport (Wloga D, Gaertig J. Post-translational modifications of microtubules. Journal of cell science 2010; 123:3447-3455; Reed N A, et al. Microtubule acetylation promotes kinesin-1 binding and transport. Curr Biol 2006; 16:2166-2172). Thus, LMWFHSA-induced microtubule acetylation appears to reduce transcytosis and basolateral trafficking in HREC. In support of this, a slight decrease in resistance across HREC monolayers following challenge with LMWFHSA was observed. While counterintuitive, it has been demonstrated that inhibition of transcytosis in endothelial cells by blocking dynamin is accompanied by an increase in paracellular permeability (Armstrong S M, et al. Co-regulation of transcellular and paracellular leak across microvascular endothelium by dynamin and Rac. Am J Pathol 2012; 180:1308-1323). Taken together with the fact that siRNA knockdown of kinesin or dynein reduces transendothelial transport of albumin (Mehta D, Malik A B. Signalling mechanisms regulating endothelial permeability. Physiological reviews 2006; 86:279-367), it seems likely that altered trafficking contributes to the observed, LMWFHSA-induced reduction in macromolecular permeability.

An intimate relationship exists between the microtubule network and the actin cytoskeleton that may link LMWFHSA-induced acetylation of α-tubulin paracellular permeability as well. The paracellular permeability observed following destabilization of microtubules can be attributed to co-localized GTPase exchange factors. GEF-H1 is released following microtubule degradation and has been shown to participate in Rho-dependent increases in vascular permeability (Bogatcheva N V, Verin A D. The role of cytoskeleton in the regulation of vascular endothelial barrier function. Microvasc Res 2008;76:202-207). Conversely, stabilization sequesters GEF-Hl as well as draws in another exchange factor, EPAC, that promotes elongation (Bogatcheva N V, Verin A D. The role of cytoskeleton in the regulation of vascular endothelial barrier function. Microvasc Res 2008;76:202-207). Therefore, the prolonged elevation in microtubule acetylation seen following treatment with LMWFHSAP could shift GTPase activity and may explain the late effect observed in TEER assays.

Mechanistically, LMWFHSA-induced acetylation of tubulin may be inversely governed by PI3-kinase and p38 MAPK. In the examples below, pharmacologic inhibition of PI3-kinase reduced while p38 inhibition synergistically enhanced acetylation following treatment. PI3-kinase stabilization of microtubules at the leading edge of fibroblasts is essential to migration (Onishi K, et al. The PI3K-Akt pathway promotes microtubule stabilization in migrating fibroblasts. Genes Cells 2007; 12:535-546). p38 inhibition counters TNFα-induced microtubule disruption in pulmonary artery endothelial cells (Petrache I, et al. The role of the microtubules in tumor necrosis factor-alpha-induced endothelial cell permeability. American Journal of Respiratory Cell and Molecular Biology 2003; 28:574-581). Interestingly, PI3-kinase inhibition increases sensitivity of tumor cells to microtubule depolymerization with vincristine (Fujiwara Y, et al. Blockade of the phosphatidylinositol-3-kinase-Akt signalling pathway enhances the induction of apoptosis by microtubule-destabilizing agents in tumor cells in which the pathway is constitutively activated. Mol Cancer Ther 2007; 6:1133-1142). This also seems to apply to other cellular functions for blockade of PI3-kinase promotes VEGF-induced activation and apoptosis in endothelial cells (Gratton J P, et al. Akt down-regulation of p38 signalling provides a novel mechanism of vascular endothelial growth factor-mediated cytoprotection in endothelial cells. J Biol Chem 2001; 276:30359-30365). Furthermore, PI3-kinase protects against ventilator-induced vascular permeability in mouse models by inhibiting p38 MAPK signalling (Peng X Q, et al. Protective role of PI3-kinase/Akt/eNOS signalling in mechanical stress through inhibition of p38 mitogen-activated protein kinase in mouse lung. Acta Pharmacol Sin 2010; 31:175-183). As a result, LMWFHSA treatment of HREC may lead to the direct inhibition of lysine-40 specific deacetylases and/or downregulation of p38 MAPK through PI3-kinase mediated cascades.

The LMWFHSA and the one or more p38 MAPK inhibitor of the present invention may be given in combination with one or more other treatments or drugs suitable for treating the disease or condition. For instance, the LMWFHSA and the one or more p38 MAPK inhibitor can be administered prior to, in conjunction with (including simultaneously with), or after the other treatment or drug. In the case of another drug, the drug and the LMWFHSA and the one or more p38 MAPK inhibitor, may be administered in separate pharmaceutical compositions or as part of the same pharmaceutical composition.

The invention also provides kits. The kits comprise a container holding LMWFHSA of the present invention as well as one or more p38 MAPK inhibitors. The kits may further comprise one or more additional containers each holding one or more other drugs suitable for use in the methods of the invention. Suitable containers include vials, bottles (including with a bottle with a dropper or a squeeze bottle), blister packs, inhalers, jars, nebulizers, packets (e.g., made of foil, plastic, paper, cellophane or another material), syringes and tubes. The kit will also contain instructions for administration of the LMWFHSA and p38 MAPK inhibitors and, optionally, the one or more other drugs suitable for use in the methods of the invention. The instructions may, for instance, be printed on the packaging holding the container(s), may be printed on a label attached to the kit or the container(s), or may be printed on a separate sheet of paper that is included in or with the kit. The packaging holding the container(s) may be, for instance, a box, or the container(s) may wrapped in, for instance, plastic shrink wrap. The kit may also contain other materials which are known in the art and which may be desirable from a commercial and user standpoint. For instance, the kit may contain instructions to help a patient manage his/her diabetes or hypertension.

As used herein, “a” or “an” means one or more.

As used herein, “comprises” and “comprising” include within their scope all narrower terms, such as “consisting essentially of” and “consisting of” as alternative embodiments of the present invention characterized herein by “comprises” or “comprising”. In regard to use of “consisting essentially of”, this phrase limits the scope of a claim to the specified steps and materials and those that do not materially affect the basic and novel characteristics of the invention disclosed herein. The basic and novel characteristics of the invention can be inhibition of vascular hyperpermeability, modulation of a cytoskeleton of an endothelial cell, or both, in an animal.

Additional objects, advantages and novel features of the present invention will become apparent to those skilled in the art by consideration of the following non-limiting examples.

### EXAMPLES

The examples below demonstrate that human retinal endothelial cells (HREC) treated with LMWF5A (“LMWFHSA”) exhibited reduced passage of horse-radish peroxidase (HRP) as well as increased trans-endothelial electrical resistance (TEER) late in culture. This was accompanied by a rapid increase in the amount and distribution of acetylated α-tubulin. Calcium depletion and inhibition of PI3-kinase reduced LMWFHSA-induced acetylation while p38 MAPK inhibition potentiated this effect.

Macromolecular permeability was evaluated by tracking the passage of horse-radish peroxidase (HRP) over 24 hours across human retinal endothelial cells (HREC) grown on porous inserts. To further explore permeability, trans-endothelial electrical resistance (TEER) was monitored for 48 hours in electrode array chambers. Additionally, the acetylation of α-tubulin was determined by immunofluorescent staining and immunoblot.

### Reagents

All reagents were purchased from Sigma Aldrich (St. Louis, Mo.) unless otherwise stated. SB203580 was obtained from ThermoFisher Scientific (Waltham, Mass.). The ≤5 kDa filtrate of commercial 5% HSA was isolated by Ampio Pharmaceuticals (Englewood, Colo.) using tangential flow filtration (TFF) and a 5 kDa MWCO Hydrosart filter membrane (Sartorius Stedim Biotech GmbH, Germany) and is also referred to as the LMWFHSA a or LMWF5A.

### Primary Retinal Endothelial Cells

Primary human retinal endothelial cells (HREC) purchased from Cell Systems (Kirkland, Wash.) were cultured in EGM-2 growth medium supplemented as recommended (Lonza, Walkersville, Md.) and used at passage 6 to 9.

Endothelial Permeability Assays HREC were grown to confluence in 0.1 μm pore transwell inserts (Thincerts; Greiner, Monroe N.C.) coated with 10 μg/cm² fibronectin. Medium containing either saline, forskolin in saline (10 μM final concentration), or LMWFHSA mixed equally with EGM-2 medium was then added. To measure macromolecular permeability, strepavidin-horseradish peroxidase (HRP; ThermoFisher Scientific) was added to the upper chambers at a final concentration of 42 ng/ml. Colorimetric analysis was evaluated after 24 hours by drawing 10 μl from the bottom chamber and mixing with 100 μL tetramethylbenzidine substrate solution (ThermoFisher Scientific). After 5 minutes, the reactions were stopped with 100 μL 0.18 M H₂SO₄ and absorbance measured at 450 nm (Spectra Max Mee microplate reader; Molecular Devices, Sunnyvale, Calif.). Resistive changes were measured by growing HREC to confluence on fibronectin coated, 8W10E+ electrode arrays attached to an ECIS Ztheta system (Applied Biophysics, Troy N.Y.) in EGM-2 medium. The solutions were then replaced with either saline or LMWF5A mixed equally with EGM-2 and impedance monitored at 4000 Hz for 48 hours with data presented as normalized resistance.

### Immunofluorescence Staining

HREC were grown on glass bottom 24-well tissue culture plates (Cellvis, Mountain View, Calif.) coated with 2% gelatin in EGM-2. The medium was then exchanged with a combination of 500 μL saline, 2× working dilutions of compounds to be tested prepared in saline, or LMWFHSA together with 500 μl EGM-2 and incubated for the indicated times. Following treatment, the cells were fixed using 10% neutral buffered formalin for 10 minutes and permeabilized with 0.1% Triton X-100 in PBS for 5 minutes. 4% goat serum (ThermoFisher Scientific) prepared in PBS was used to block the cells for 1 hour then the cells were exposed to anti-acetylated α-tubulin clone 6-11B-1 antibody (1:1000; Santa Cruz Biotechnology, Santa Cruz, Calif.) in blocking solution overnight at 4° C. Alexa fluor 488 conjugated anti-mouse IgG (1:1000; Invitrogen, Carlsbad, Calif.) was then added for 1 hour followed by DAPI counter staining (300 nM in PBS; ThermoFisher Scientific) for 5 minutes. Randomly selected frames were photographed on an inverted microscope (Zyla sCMOS camera; Andor, South Windsor, Conn. and eclipse Ti; Nikon, Melville, N.Y.) and fluorescent intensity measured using ImageJ software (http://imagej.nih.gov/ij) (Schneider C A, et al. NIH Image to ImageJ: 25 years of image analysis. Nature methods 2012; 9:671-675). For normalization, the number of DAPI staining nuclei was determined for each frame and data presented as median FU/DAPI objects.

Immunoblot Analysis

HREC were grown to confluence on 2% gelatin coated 6-well culture dishes then treated as described for immunofluorescent staining with volumes scaled accordingly. Following treatment, cells were lysed in 100 μl lysis buffer (Qproteome Mammalian Protein kit; Qiagen, Valencia, Calif.) according to manufacturer's instructions and cleared by cooled, 4° C. centrifugation at 12,000×g for 10 min. The lysates were separated by SDS-PAGE after boiling in Bolt Reducing Buffer and Bolt LDS Sample Buffer (ThermoFisher Scientific). Western blot analysis was performed using a mouse anti-acetylated α-tubulin clone 6-11B-1 and goat anti-actin antibody mix (1:1000; Santa Cruz Biotechnology, Santa Cruz, Calif.) followed by a chicken Alexa fluor 594 conjugated anti-mouse IgG and chicken Alexa fluor 488 conjugated anti-goat IgG antibody mix (1:1000; Invitrogen, Carlsbad, Calif.). Immunoblots visualized on animage station (Carestream Health, Rochester, N.Y.) with appropriate filter sets.

### Data Analysis

One-way ANOVA tests were performed with post hoc Bonferroni correction and 95% confidence intervals constructed using Excel with significance set at 0.05 (Microsoft; Redmond, Wash.).

### Example 1

### Effect of LMWFHSA on Retinal Endothelial Cell Permeability

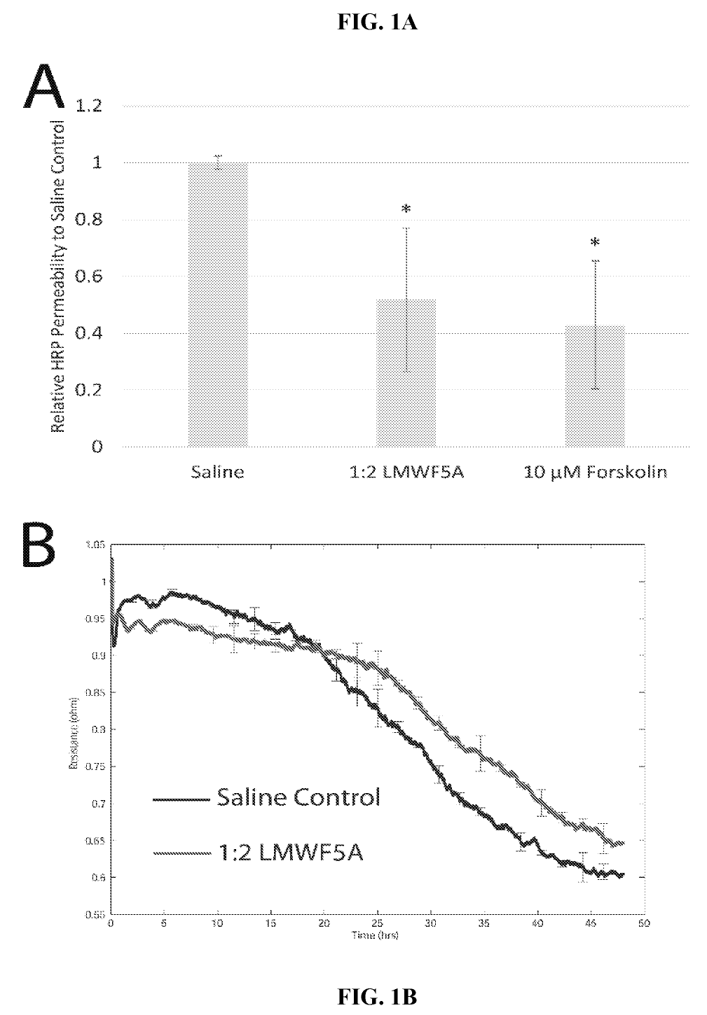

To evaluate the effect of LMWFHSA on vascular permeability, two in vitro assays were employed. In the first, passage of HRP was determined across confluent monolayers of HREC established on porous trans-well inserts. As seen in FIG. 1A, LMWFHSA (“LMWF5A”) significantly reduced HRP permeability in this model by 48% as compared to saline treated controls (p<0.025; n=3). A similar reduction was achieved by treatment with 10 μM forskolin.

Having established that LMWFHSA decreases macromolecular permeability, trans-endothelial electrical resistance was then monitored for 48 hours following treatment. In this assay, an immediate increase in resistance was observed after exposure to LMWFHSA, lasting 30 minutes, with a subsequent reduction of 2-5% for approximately 15 hours as compared to saline (FIG. 1B). After 24 hours, however, LMWFHSA treated cells exhibit an increase in resistance that persists to the completion of the experiment. Taken together, these data suggest that LMWFHSA treatment initially reduces transcytosis, then offers protect against the breakdown of barrier function that corresponds with medium exhaustion.

### Example 2

### LMWFHSA Induces Temporal and Phenotypic Changes in HREC α-Tubulin Acetylation

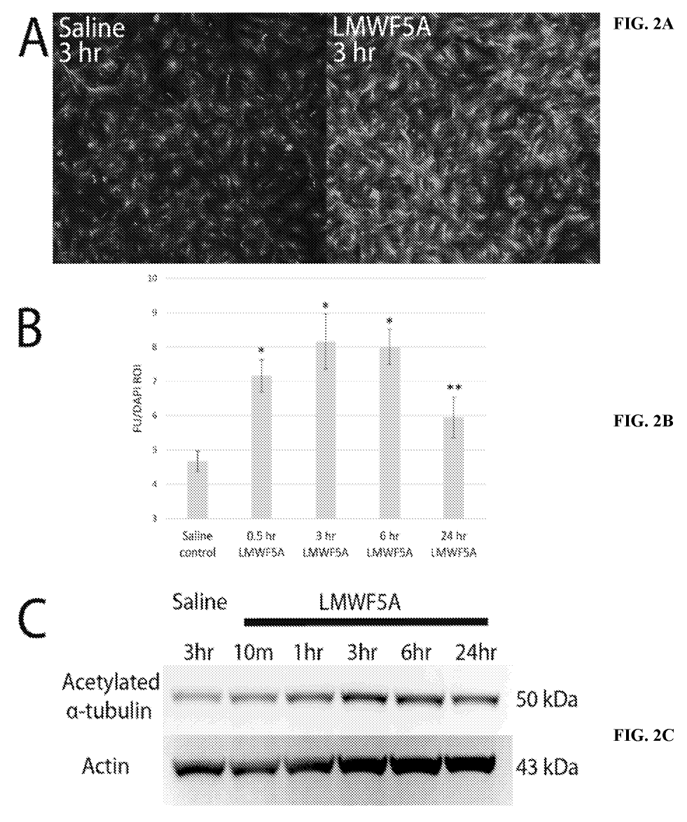

Previous studies demonstrated that LMWFHSA treatment of bone marrow derived mesenchymal stem cells resulted in a reduction of cytoplasmic stress-fibers concurrent with the development of filopodia-like projections around the periphery of the cell (Bar-Or D, et al. Low Molecular Weight Fraction of Commercial Human Serum Albumin Induces Morphologic and Transcriptional Changes of Bone Marrow-Derived Mesenchymal Stem Cells. Stem Cells Transl Med 2015; 4:945-955). In HREC, however, no appreciable change in f-actin was observed following treatment (data not shown). Instead, immunofluorescence (IF) staining revealed that 3 hours after exposure to LMWFHSA, HREC exhibit a marked increase in α-tubulin acetylation; a perceived marker of microtubule stabilization (FIG. 2A). FIG. 2B depicts a representative IF experiment in which temporal changes in LMWFHSA-induced tubulin acetylation were tracked. A significant increase (p<0.01; n=6) over the saline control was observed at all time points tested: 1.5-fold at 30 minutes, 1.8-fold at 3 hours, 1.7-fold at 6 hours, and 1.3-fold at 24 hours. These findings were confirmed by western blot analysis (FIG. 2C). In addition, this technique afforded the sensitivity to detect increased acetylation manifesting after 10 minutes.

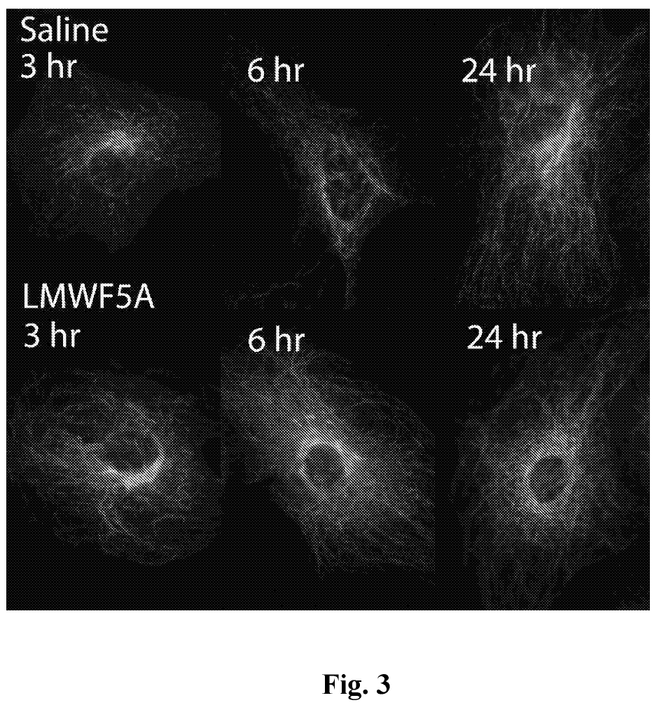

IF also showed that LMWFHSA alters the distribution of acetylated tubulin in HREC (FIG. 3). When viewed at higher magnification, acetylated α-tubulin in saline controls is primarily located in microtubule organizing centers around the nucleus. In contrast, LMWFHSA treated HREC exhibit elevated cytoplasmic and perinuclear staining.

### Example 3

### LY294002 (a PI3-Kinase Inhibitor) Reduces LMWFHSA Induced Acetylation of α-tubulin, While SB203580 (a p38 MAPK Inhibitor) Potentiates LMWFHSA-Induced Acetylation of α-tubulin

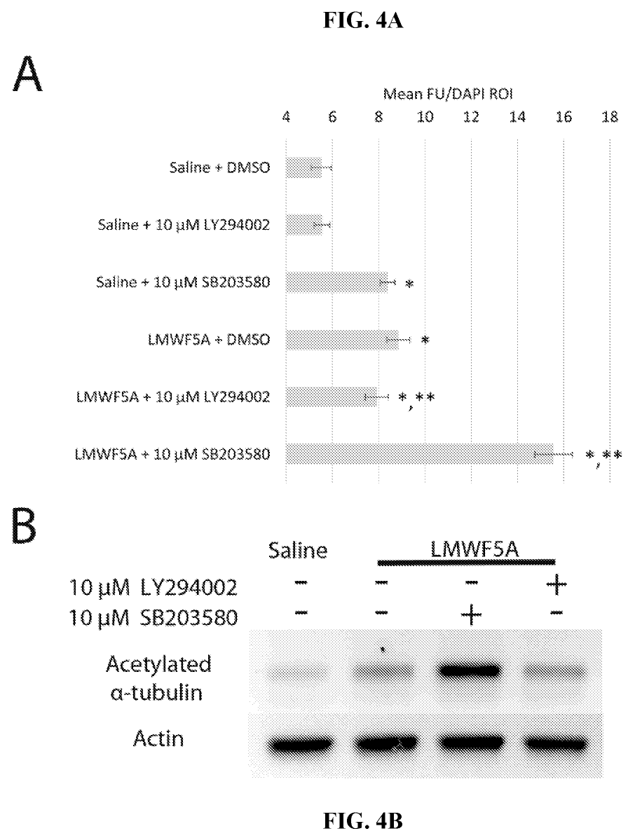

This example evaluates the effect of inhibition of PI3-kinase and inhibition of p38 MAPK on LMWF5A-induced α-tubulin acetylation of HREC. HREC were treated with LMWFHSA in the presence of specific inhibitors and IF was performed after 3 hours. As seen in FIG. 4A, inhibition of PI3-kinase with 10 μM LY294002 reduced LMWFHSA-induced acetylation (p<0.025 vs LMWF5A+DMSO; n=6). When percent inhibitions were calculated for four separate experiments performed in triplicate, it was found that LY294002 reduced LMWFHSA-induced acetylation by 24% (95% CI 29-19). Conversely, inhibition of p38 MAPK with SB203580 dramatically increased α-tubulin acetylation versus both saline-DMSO controls (p<0.01; n=6) and LMWF5A-DMSO (p<0.025; n=6) treated cells by 57% (95% CI 63-51) and 222% (95% CI 236-208) respectively. A similar pattern emerged when analyzed by western blot (FIG. 4B).

All of the documents cited herein are incorporated herein by reference.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

1. A method of inhibiting vascular hyperpermeability in an animal in need thereof comprising administering to the animal an effective amount of
(i) a pharmaceutical composition prepared by removing albumin from a human serum albumin composition; and
(ii) one or more p38 MAPK inhibitors.

2. The method of claim 1 wherein the one or more p38 MAPK inhibitors is selected from the group consisting of SB 203580, SB 203580 hydrochloride, SB 202190, SB 239063, SB 706504, AL 8697, AMG 548, CMPD-1, DBM 1285 dihydrochloride, EO 1428, JX 401, ML 3403, RWJ 67657, SCIO 469 hydrochloride, SKF 86002 dihydrochloride, SX 011, TA 01, TA 02, TAK 715, VX 702, VX 745, p38 MAPK Inhibitor TOCRISET™, and combinations thereof.

3. The method of claim 1 wherein the animal has a disease or condition mediated by vascular hyperpermeability.

4. The method of claim 3 wherein administration of the pharmaceutical composition and the one or more p38 MAPK inhibitors is commenced immediately upon diagnosis of the disease or condition.

5. The method of claim 3, wherein the disease or condition is an ocular disease.

6. The method of claim 3 wherein the disease or condition is a vascular complication of diabetes.

7. The method of claim 6 wherein the vascular complication is edema, accumulation of low density lipoproteins in subendothelial space, accelerated atherosclerosis, accelerated aging of vessel walls in the brain, myocardial edema, myocardial fibrosis, diastolic dysfunction, diabetic cardiomyopathy, retardation of lung development in the fetuses of diabetic mothers, alterations of one or more pulmonary physiological parameters, increased susceptibility to infections, vascular hyperplasy in the mesentery, diabetic neuropathy, diabetic macular edema, diabetic nephropathy, diabetic retinopathy, or redness, discoloration, dryness and ulcerations of the skin.

8. The method of claim 7 wherein the vascular complication is edema.

9. (canceled)

10. (canceled)

11. (canceled)

12. The method of claim 7 wherein the vascular complication is diabetic retinopathy.

13. (canceled)

14. (canceled)

15. The method of claim 3 wherein the disease or condition is an acute lung injury, acute respiratory distress syndrome, age-related macular degeneration, atherosclerosis, choroidal edema, choroiditis, coronary microvascular disease, cerebral microvascular disease, diabetes, Eals disease, edema caused by injury, edema associated with hypertension, glomerular vascular leakage, hemorrhagic shock, hypertension, Irvine Gass Syndrome, ischemia, macular edema, nephritis, nephropathies, nephrotic edema, nephrotic syndrome, neuropathy, organ failure due to edema, pre-eclampsia, pulmonary edema, pulmonary hypertension, renal failure, retinal edema, retinal hemorrhage, retinal vein occlusion, retinitis, retinopathy, silent cerebral infarction, systemic inflammatory response syndrome, transplant glomerulopathy, uveitis, vascular leakage syndrome, vitreous hemorrhage or Von Hipple Lindau disease.

16. The method of claim 15 wherein the disease or condition is a macular edema.

17. (canceled)

18. (canceled)

19. The method of claim 1 wherein the animal is in need of the pharmaceutical composition and the one or more p38 MAPK inhibitors, because of one or more early signs of, or a predisposition to develop, a disease or condition mediated by vascular hyperpermeability.

20. The method of claim 19 wherein the disease or condition is diabetes, hypertension, atherosclerosis or an ocular disease.

21. The method of claim 1 wherein the vascular hyperpermeability is vascular hyperpermeability of a continuous endothelium found in, or around, a brain, diaphragm, duodenal musculature, fat, heart, kidney, large blood vessel, lung, mesentery, nerve, retina, skeletal muscle, skin or testis.

22. The method of claim 21 wherein the continuous endothelium is found in, or around, a brain, heart, lung, nerve or retina.

23. (canceled)

24. (canceled)

25. The method of claim 1, wherein the step of removing the albumin comprises treating the human serum albumin composition by a separation method selected from the group consisting of ultrafiltration, sucrose gradient centrifugation, chromatography, salt precipitation, and sonication.

26. The method of claim 25, wherein the step of removing comprises passing the human serum albumin composition over an ultrafiltration membrane with a molecular weight cut off that retains the albumin, and wherein the resulting filtrate comprises DA-DKP.

27. A pharmaceutical composition comprising a composition prepared by removing albumin from a human serum albumin composition and one or more p38 MAPK inhibitors for the treatment of a disease or condition mediated by vascular hyperpermeability and/or for the inhibition of vascular hyperpermeability.

28. The composition of claim 27 wherein the one or more p38 MAPK inhibitors is selected from the group consisting of SB 203580, SB 203580 hydrochloride, SB 202190, SB 239063, SB 706504, AL 8697, AMG 548, CMPD-1, DBM 1285 dihydrochloride, EO 1428, JX 401, ML 3403, RWJ 67657, SCIO 469 hydrochloride, SKF 86002 dihydrochloride, SX 011, TA 01, TA 02, TAK 715, VX 702, VX 745, p38 MAPK Inhibitor TOCRISET™, and combinations thereof.

29. The composition of claim 27, wherein the disease or condition is an ocular disease.
